(19) United States
(12) Plant Patent Application Publication
(10) Pub. No.: US 2026/0314511 P1
(43) Pub. Date: Oct. 8, 2026

(54) STRAWBERRY PLANT NAMED 'NSG 48'

(50) ;

(71) Applicant: Nova Siri Genetics S.R.L., Policoro (IT)

(72) Inventor: Nicola TUFARO, Policoro (IT)

(73) Assignee: Nova Siri Genetics S.R.L., Policoro (IT)

(21) Appl. No.: 19/549,315

(22) Filed: Feb. 25, 2026

(30) Foreign Application Priority Data
Apr. 2, 2025 (QZ) 2025/0650

(51) Int. Cl. A01H 6/74; A01H 5/08

(52) U.S. Cl. CPC A01H 6/7409; A01H 5/08
USPC PLT/208

(57) ABSTRACT

A new and distinct variety of strawberry plant, referred to by its cultivar name, ‘NSG 48’, is described. The new plant variety forms white inflorescence. The growth habit is semi-erect. Additionally, the new variety produces fruits which are medium to large in size, medium-red in colour, mainly conical in shape, with very firm flesh, and moderately longer than wide.

Latin name of genus and species of plant claimed: Fragaria x ananassa Duchesne ex Rozier.

Variety denomination: ‘NSG 48’.

### CROSS REFERENCE TO RELATED APPLICATIONS

This application claims priority to Plant Breeders' Right Application No. 2025/0650, which was filed at Community Plant Variety Office (CPVO) in the European Union on Apr. 2, 2025, the contents of which are hereby incorporated by reference for all purposes.

### BACKGROUND OF THE INVENTION

The new and distinct short-day strawberry plant variety of the present invention was the product of a controlled breeding program conducted in Policoro (MT), Italy, located at 40° 197 N-16° 656 E. The new plant variety is the result of an uncontrolled cross. The female parent (i.e., the seed-bearing parent) is the ‘Charlene’ variety (U.S. Plant Pat. No. 28,220), which is a short-day strawberry plant variety with medium-early flowering and fruiting and that produces attractive, medium-red coloured fruits with high sugar content. The male parent (i.e., the pollen parent) is unknown.

The parentage can be summarized as follows:
‘Charlene’ x Unknown Parent

The seeds resulting from the above uncontrolled cross were sown, and small plants were obtained that were physically different from each other. Selective study and analysis made it possible to identify a single short-day strawberry plant of the new variety.

The new plant variety has undergone asexual propagation in a nursery through the use of stolons in Ladbergen, Germany, located at 52° 07′41.6″N-7° 45′29.0″ E, and in Policoro (MT), Italy, by using stolons and in vitro tissue culture. No root decomposition problems were observed during cultivation. The combination of characteristics presented by the new plant variety proved to be stable and reliably transmitted to subsequent generations after such asexual reproduction.

Therefore, the characteristics of the new plant variety remain unaltered after asexual reproduction.

### SUMMARY OF THE INVENTION

The new strawberry plant of the present invention possesses the following combination of characteristics:
(a) displays a medium-dense density of the foliage and semi-erect growth habit,
(b) provides medium-early and white inflorescence, generally above the level of the foliage,
(c) commonly exhibits a calyx that is wider than the diameter of the corolla when it is open, and
(d) produces abundant, attractive, medium-early ripening fruits that are medium to large in size, medium-red in colour, mainly conical in shape, with very firm flesh, and moderately longer than wide.

The new plant variety meets the needs of the horticultural industry reasonably well. The new plant variety has characteristics that are commonly pursued by commercial strawberry growers. For example, the new plant variety offers good yields of medium-red fruit with medium-early ripening. Therefore, the new plant variety is suitable for fresh consumption and it is considered promising for commercial introduction. The new plant variety requires an induction period for flowering. This can be achieved by growing it in a colder climate away from the equator or at a higher altitude above sea level.

The new plant variety can be easily distinguished from its known progenitor. More specifically, the variety ‘Charlene’ (i.e., the seed-bearing parent) has a coloration of the underside of the leaf that is a color between Greyed-Green Group 191B and 191C, which differs from the coloration of the underside of the leaf of the new plant variety, which is a color between Green Group 138B and 138C. In addition, the sepals of ‘Charlene’ are generally narrower, with a lower surface colour of Yellow-Green Group 147B, clearly distinguishable from the sepals of the new plant variety, which are wider and present a lower page coloration that is Green Group 139C. Also, the ‘Charlene’ variety generally produces fruit with a darker external colour, compared to fruit produced by the new plant variety. The colour of the internal flesh of ‘Charlene’ is also darker than that of the new variety. The diameter of the calyx of the new variety's fruit is larger than the diameter of the calyx of ‘Charlene’'s fruit.

The new plant variety can be easily distinguished from other similar varieties. Compared to the new plant variety, for example, the variety ‘Melissa’ (U.S. Plant Patent No. 28,081) has less shiny leaves, a larger diameter of the flower, and a bigger fruit that is much longer than wide. The new plant variety also differs from ‘ROCIERA FNM’ (European Union Community Plant Variety Rights No. 46254) because the new plant variety has much rougher and shinier leaves. The colour of the fruit of ‘ROCIERA FNM’ is orange-red, with the flesh and core being much lighter in colour than the new plant variety, which is medium-red both inside and outside.

The new variety has been named ‘NSG 48’ and initially designated as qd20.48.

### BRIEF DESCRIPTION OF THE PHOTOGRAPHS

The accompanying photographs show, as realistically as is reasonably possible in colour illustrations, strawberry plants and botanical parts of the new variety identified with the code qd20.48.

The strawberry plants of the new variety illustrated were reproduced asexually through the use of stolons, in a nursery in Ladbergen, Germany, located at 52° 07′41.6″N-7° 45′29.0″ E, and planted under the cover of plastic tunnels around mid-October 2024 in Policoro (MT)-Italy, located at 40° 197 N-16° 656 E.

FIG. 1—group of plants with fruit of the new variety, in February 2025.

FIG. 2—individual plants of the new variety where it is possible to observe a semi-erect and medium-dense configuration.

FIG. 3—specimen of upper surface of the new plant variety's typical trifoliate leaf, in which the petiole and stipules are also present. Dimensions are given in centimeters and inches.

FIG. 4—specimen of under surface of the new plant variety's typical trifoliate leaf and leaf ribs; the petiole and stipules are also present. Dimensions are given in centimeters and inches.

FIG. 5—specimen of upper surface of the new plant variety's terminal leaflet.

FIG. 6—specimen of under surface of the new plant variety's terminal leaflet.

FIG. 7—fruiting specimen of the new plant variety. Dimensions in centimeters and inches are included.

FIG. 8 flowering specimen of the new plant variety. Dimensions in centimeters and inches are included.

FIG. 9 specimen of flowers of the new plant variety, seen from above. Dimensions in centimeters and inches are included.

FIG. 10 specimen of the new plant variety's calyxes, seen from above. Dimensions in centimeters and inches are included.

FIG. 11 specimen of the new plant variety's mature fruit-whole. Dimensions in centimeters and inches are included.

FIG. 12 specimen of the new plant variety's mature fruit-transverse and longitudinal section. Dimensions in centimeters and inches are included.

FIG. 13 specimen of the new plant variety's stipules-open. Dimensions in centimeters and inches are included.

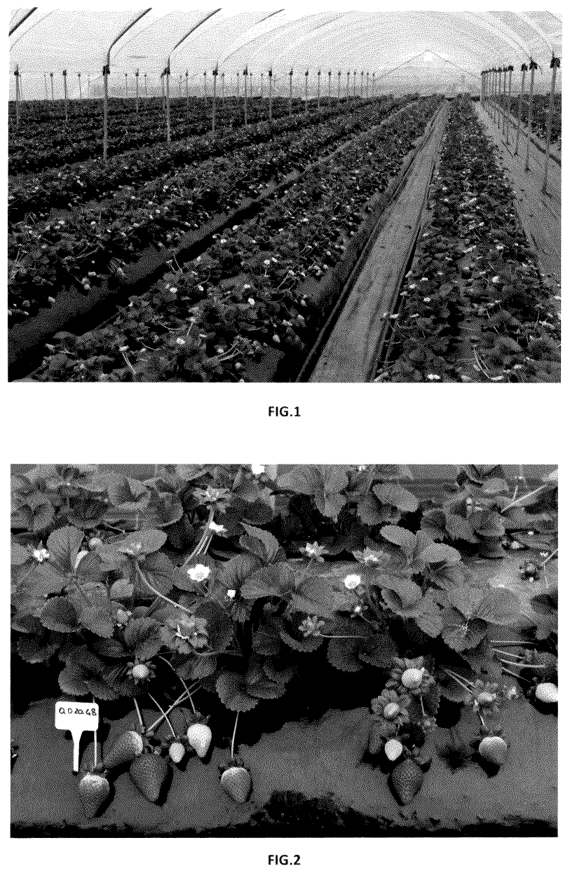

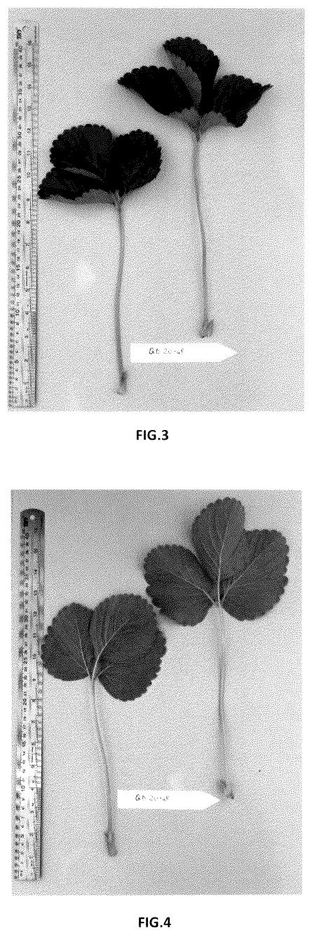

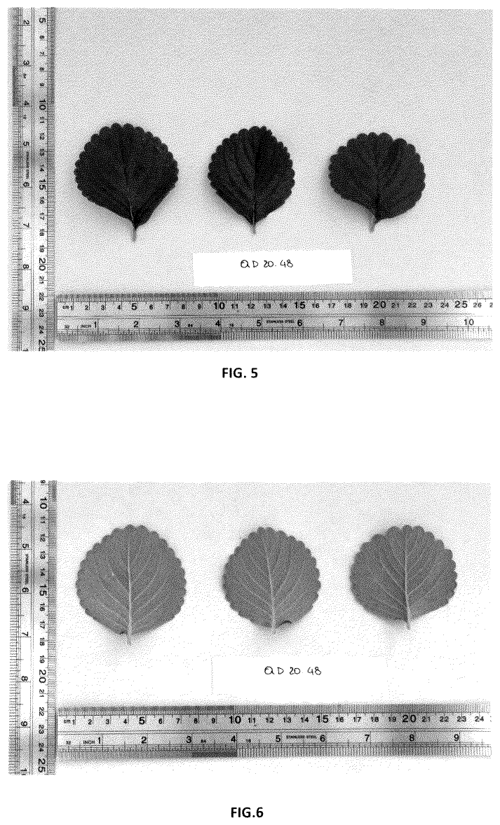

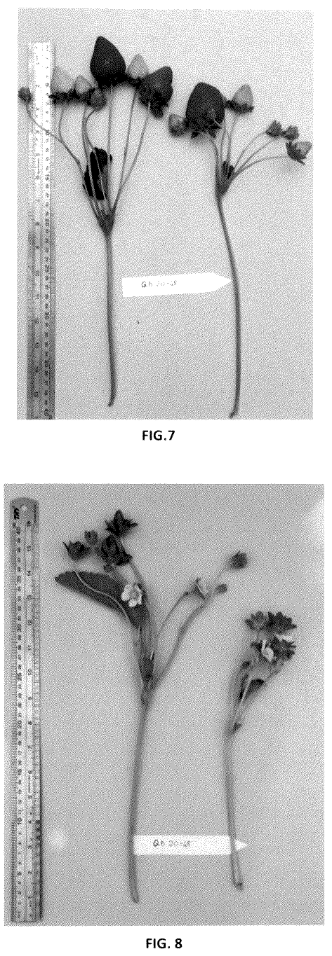

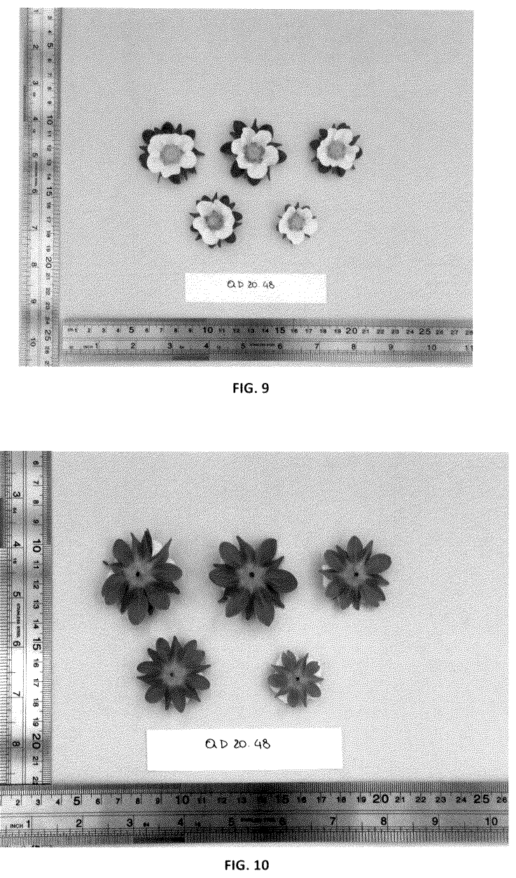

### DETAILED BOTANICAL DESCRIPTION

The colours referenced herein were identified using The Royal Horticultural Society (“R.H.S.” Colour Chart, London, England, Edition V). The colour chart code is associated with the corresponding chromatic subgroup. The R.H.S. Colour Chart designation used herein represents the closest colour observed on the majority of the specified botanical features.

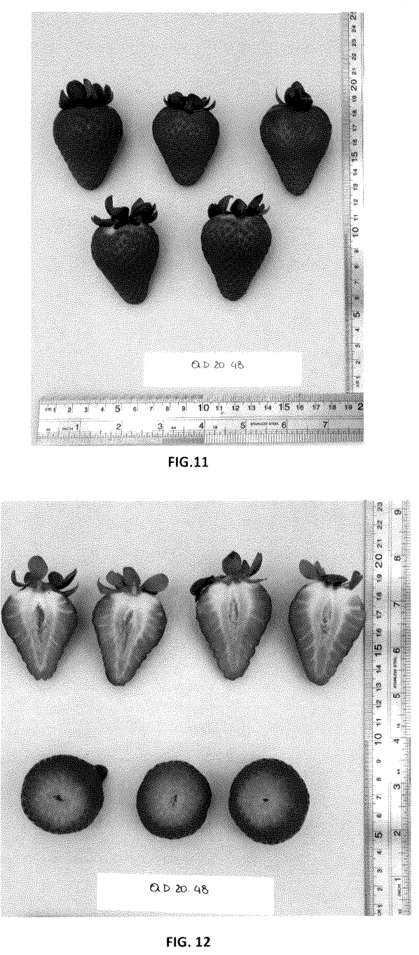

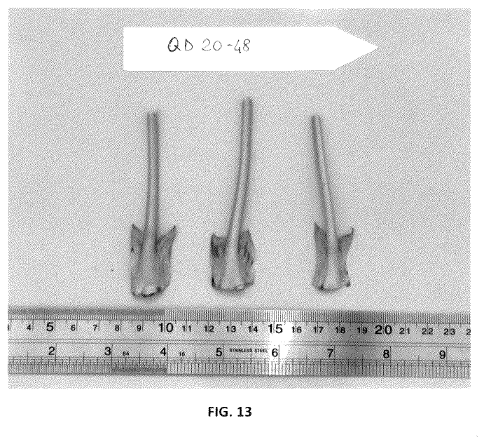

The detailed botanical description is based on plants of the new variety that have been reproduced asexually through the use of stolons in Ladbergen, Germany, located at 52° 07′41.6″N-7° 45′29.0″E, transplanted in Policoro (MT), Italy, located at 40° 197 N-16° 656 E in October and grown under the cover of plastic tunnels. The age of the plants of the new variety on which the botanical description was made is between two and ten months from the date of transplantations.
Plant:
   Type.—Short-day.
   Configuration.—Semi-erect and medium-dense.
   Height (average).—From approximately 23 cm to 29 cm.
   Leaf.—Length: From approximately 28 cm to 36 cm, including petiole and stipule. — width: from approximately 12 cm to 16 cm.
   Leaflets.—Number: commonly 3. — terminal leaflet size: medium to large, approximately 8.5 cm long on average and approximately 7.8 cm wide on average. — terminal leaflet: equal in length and width or slightly longer than wide, it has a concave cross-section, a serrate, crenate margin, and a rounded base. — blistering: commonly strong. — glossiness of the upper surface: medium to strong. — colour: there is no variegated colouration; the upper surface colouration is Green Group 137A; that of the lower surface is Green Group 138B. — texture of the upper surface: commonly wrinkled with small translucent waxy points and few trichomes on the surface, with a quite vertical orientation. — texture of the under surface: generally wrinkled with trichomes mainly present on the ribs. — venation pattern: reticulate. — foliage: medium to dense.
   Stolons.—Approximately 30 stolons per plant on average, coloration is Yellow-Green Group 144C, medium-weak anthocyanin coloration is a colour between Red-Purple Group 59C and 60C, medium pubescence. The distance between plants rooted in the same stolon is approximately 29 cm to 37 cm on average.
   Petioles.—Approximately from 18 cm to 24 cm in length on average (measured from the base of the stipules). In sections, the average of the minor axis and the average of the major axis, measured at approximately 3 cm from the base of the stipule, are approximately 3.6 mm and 5.3 mm, respectively. The colour is Yellow-Green Group N144D, and they commonly show a fine pubescence that is horizontally disposed.
   Stipules.—Approximately 2.8 cm and 3.5 cm in length on average, average width of closed stipules, measured at the base of the stipule, is approximately 1.4 cm. The average width of open stipules is approximately 2 cm. The colour is a colour between Yellow-Green 145B and 145C with an anthocyanin coloration between Red-Purple Group 58B and 58C.
   Floral fragrance.—Present.
Inflorescence:
   Flowering time.—Mid-early, around the third week of December in Policoro (MT), Italy, located at 40° 197 N-16° 656 E.
   Flower disposition.—Above the level of the foliage.
   Flowers number.—Many, about 10.
   Pedicel hairs.—Pubescence generally disposed outwards.
   Pedicel colour.—Yellow-Green Group N145A.
   Pedicel length.—The average length of the main and secondary pedicels measured from the base of the calyx is approximately 13 cm.
   Pedicel diameter.—On average, the diameter measured at approximately 2 cm from the calyx is around 2.2 mm.
   Flower size.—Medium, with an average diameter of primary flowers (measured without calyx) of approximately 3 cm and an average diameter of secondary flowers of approximately 2 cm. The open calyx is slightly larger than the diameter of the corolla, on average it has a diameter of approximately 3.6 cm for primary flowers.
   Petals.—Number: on average between 6 and 7, typically 7. — arrangement and shape: slightly overlapping, moderately shorter in length than wide. — size: approximately 1.4 cm on average in width and approximately 1.2 cm in length. — colour: White Group NN155B on the upper surface. — texture of the upper surface: generally spongy texture, smooth and glabrous surface, with small translucent points; slight roughness at the level of insertion on the calyx. — texture of the under surface: generally slight roughness, becoming more pronounced at the level of insertion on the calyx, glabrous surface.
   Stamens.—Average number: approximately 26. — average length: between approximately 2 mm and 5 mm. — filament colour: Yellow Group 13A.
   Anthers.—Number: approximately 26 on average, and colour is Yellow-Green Group 154C.
   Pollen.—In abundance. — colour: Yellow-Orange Group 14A.
   Stigma.—Shape: jagged-fan. — size: average openness of approximately 154 μm. — texture: waxed stigma.
   Style.—Average length: approximately 702 μm. — average diameter: approximately 105 μm, which considerably thins at the level of insertion on the ovary, entirely crossed by a stylar canal with an average diameter of approximately 21 μm.
   Ovary.—Shape: oval, with a major axis and minor axis measuring approximately 328 μm and 234 μm, respectively.
   Sepals.—Shape: generally lanceolate in configuration; the attitude is commonly upwards, and in general, slightly extend beyond the petals. — number: on average between 11 and 12. — size: between approximately 1.6 cm and 1.8 cm in length on average and approximately 0.8 cm and 1 cm in width on average, at the broadest point. — colour: the upper surface is a colour between Green Group 143A and 138A, and the lower surface is Green Group 139C. — texture of the upper surface: usually slightly wrinkled. — texture of the under surface: generally, slightly wrinkled with few trichomes on the entire surface and especially at the base.
Fruit:
   Bearing.—Non-remontant.
   Timing.—Early to medium fruiting.
   Shape.—Conical, moderately longer than wide, with a slight difference between the terminal fruits and the others.
   Size.—Medium, with an average length of the primary fruit between approximately 4.8 cm and 5.2 cm; between approximately 3.5 cm and 3.9 cm in width on average, at the broadest point, and an average weight of approximately 28 grams.
   Surface.—Generally smooth with a strong glossiness.
   External colour.—Substantially uniform and a colour between Red Group 43A and 44A.
   Internal colour.—The flesh is a colour between Orange-Red Group N30A and 30A and the core is Orange-Red Group 33A.
   Firmness.—Very good.
   Cavity.—Small to medium, as shown in FIG. 12.
   Achenes.—Generally located below the surface of the fruit, covering almost the entire surface of the fruit with only a narrow band where the achenes are absent. The colour is a color between Red Group 46A and Red Group 46B when the fruit is fully ripe.
   Calyx.—Commonly at the same level as the fruit at the point of attachment. The calyx commonly attaches to the fruit with medium adherence, the sepals are generally disposed slightly upwards, and the diameter of the calyx in relation to the diameter of the fruit is commonly equal.
   Peduncle.—Long, elliptical in cross-section, with a medium length of the peduncle, measured from the base of the calyx of the primary fruit, between approximately 28 cm and 39 cm. The peduncle measured includes the pedicel bearing the main fruit in addition to the underlying peduncle. The average minor axis and average major axis in cross-section are approximately 3.9 mm and 4.6 mm, respectively. The diameters of the axes were measured at approximately 3 cm from the origin of the peduncle. The colour is a color between Yellow-Green Group 144B and N144C.
   Pedicel.—Commonly with pubescence extending outwards, and the colour is Yellow-Green Group N144D.
   Storability.—On average 6 to 7 days the fruit can be stored in cold storage.
   Shipping quality.—High, qualitative parameters are shown in Table 4.
   Market use.—Fresh.
Development:
   Fertilization.—Self-fertile.
   Diseases tolerance.—During the observation period and at the date, no sensitivities to any disease were observed.
   Winter hardness/cold tolerance.13 Unknown.
   Drought/heat tolerance.—Good.

Plants of the variety ‘NSG 48’ have not been observed under all possible environmental conditions. Therefore, it is possible that the phenotypic expression may vary depending on the temperature, duration and intensity of light, cultivation practices, and other environmental conditions.

### ADDITIONAL COMPARATIVE DATA

Additional comparative data for the new plant variety are provided below: ‘NSG 48’, ‘Charlene’ (i.e., the female parent), and ‘Melissa’ and ‘ROCIERA FNM’ varieties.

The plants of the above varieties, obtained by asexual reproduction in the nursery, using stolons in the spring of 2024, were then transplanted in Policoro, (MT), Italy, located at 40° 197 N-16° 656 E, around mid-October 2024. All varieties were grown under the cover of plastic tunnels in Policoro (same geolocation). The first fruit harvest took place on Jan. 9, 2025, and the last on May 20, 2025. The fruit was analyzed and compared on the dates indicated. The average data are the following:

TABLE 1
Cumulative production of top-quality fruit (g/plant)

| variety | Feb. 28th | Mar. 30th | Apr. 30th | May 20th |
|---|---|---|---|---|
| ‘NSG 48’ | 46 | 231 | 418 | 581 |
| ‘Charlene’ | 37 | 148 | 373 | 560 |

TABLE 2
Overall comparison of the average fruit weight in
Policoro, Italy, during the year 2025

| plant | g/fruit |
|---|---|
| ‘NSG 48’ | 24 |
| ‘Charlene’ | 22 |
| ‘Melissa’ | 27 |
| ‘ROCIERA FNM’ | 24 |

TABLE 3
Average fruit weight on specific dates in 2025

| | Feb. 28th | Mar. 30th | Apr. 30th | May 20th |
|---|---|---|---|---|
| variety | (grams) | (grams) | (grams) | (grams) |
| ‘NSG 48’ | 27 | 25 | 23 | 21 |
| ‘Charlene’ | 23 | 22 | 23 | 20 |
| ‘Melissa’ | 30 | 29 | 23 | 26 |
| ‘ROCIERA FNM’ | 28 | 26 | 21 | 21 |

TABLE 4
Fruit analyzed in 2025

| | | | | ‘ROCIERA |
|---|---|---|---|---|
| | ‘NSG 48’ | ‘Charlene’ | ‘Melissa’ | FNM’ |
| Firmness | 0.81 | 0.70 | 0.80 | 0.78 |
| (average)* | | | | |
| Dry matter (%)** | 9.64 | 9.92 | 9.88 | 7.91 |
| pH (to 20º) | 3.53 | 4.33 | 4.61 | 3.77 |
| Acidity as | 0.98 | 0.80 | 0.76 | 0.76 |
| anhydride | | | | |
| citric (%)*** | | | | |
| Soluble solids | 10.0 | 9.10 | 8.30 | 9.0 |
| (°Brix) **** | | | | |
| Maturity | 10.2 | 11.4 | 10.9 | 11.9 |
| index***** | | | | |

*Penetration resistance measured in kilograms using a PCE Instruments PCE-FM 200 penetrometer.
**MIP 400 2024 rev.00.
***D.M. 3 Feb. 1989 GU N.168 20 Jul. 1989 Met. 15
**** MIP 402 2024 Rev.00
*****MIP 160 2013 Rev.0

I claim:

1. A new and distinct variety of strawberry plant named ‘NSG 48’ substantially as illustrated and described herein.
